(19) United States
(12) Patent Application Publication
(10) Pub. No.: US 2020/0268063 A1
(43) Pub. Date: Aug. 27, 2020

(54) SMART GARMENT, SMART WEARABLE DEVICE AND SMART WEARABLE ASSEMBLY

(71) Applicant: Chia-Chieh HUNG, Taoyuan City (TW)

(72) Inventor: Chia-Chieh HUNG, Taoyuan City (TW)

(21) Appl. No.: 16/412,985

(22) Filed: May 15, 2019

(30) Foreign Application Priority Data
Feb. 27, 2019 (TW) 108202456

(51) Int. Cl. A41D 1/00; A61B 5/0205; A61F 7/00; A61H 7/00; A61B 5/00; A63B 24/00

(52) U.S. Cl. CPC A41D 1/005; A61B 5/02055; A61F 7/00; A61H 7/001; A61B 5/6803; A61B 5/021; A61B 5/6807; A61B 5/681; A61B 5/749; A63B 24/0075; A61B 5/6806

(57) ABSTRACT

A smart garment having: a garment; at least one node device disposed on the garment; and a connecting device disposed on the garment and having a connecting port electrically coupled with the at least one node device and a connection interface for coupling with a wearable device or a mobile device, such that the wearable device or the mobile device can utilize the connection interface to obtain sensed information of the at least one node device, or drive the at least one node device to change a physical quantity applied on the garment.

### BACKGROUND OF THE INVENTION

### Field of the Invention

The present invention relates to a smart wearable assembly, and more particularly to a smart wearable assembly that allows a smart wearable device to be detached from a smart garment to prevent damaging the wearable device when the smart garment is washed.

### Description of the Related Art

A typical smart garment has one or more sensing elements and a micro processing unit installed on a garment so that the micro processing unit can record a wearer's physiological information via the sensing elements.

However, when the smart garment needs washing, how to prevent the micro processing unit from being damaged by moisture is a very difficult problem. In order to protect the micro-processing unit, it generally takes a high cost on the waterproof mechanism of the micro-processing unit.

To solve the above problems, a novel smart wearable assembly is needed.

### SUMMARY OF THE INVENTION

One objective of the present invention is to provide a smart wearable assembly that a wearable device can be attached to or removed from a smart garment via a connection device on the smart garment to prevent damaging the wearable device when the smart garment is washed.

Another objective of the present invention is to provide a smart wearable assembly that can sense information such as position, heart rate, blood pressure, temperature or acceleration by a smart garment for monitoring a wearer's exercise history or body condition.

Still another objective of the present invention is to provide a smart wearable assembly that can apply heat or pressure to a wearer by a smart garment to provide a function of relaxing, protecting or warming the wearer's body.

To attain the above objectives, a smart garment is proposed including:

a garment;

at least one node device distributed on the garment, and the node device being a fixed device or a detachable device; and

a connecting device disposed on the garment and having a connecting port electrically coupled to the at least one node device, and a connection interface for coupling to a wearing device or a mobile device so that the wearing device or the mobile device can utilize the connection interface to obtain sensed information of the at least one node device, or drive the at least one node device to change a physical quantity applied on the garment.

For possible embodiments, the sensed information may include position, heart rate, blood pressure, temperature, or acceleration.

For possible embodiments, the physical quantity may include heat or pressure.

For possible embodiments, the connection interface can be a wired interface or a wireless interface.

For possible embodiments, the garment may include at least one element of a group consisting of a top, pants, a jumpsuit, a glove, a sock, a hat, a headgear, and a coat.

Besides, the present invention further provides a smart wearable device having a communication interface for coupling with the connection interface of the smart garment to obtain the sensed information of the at least one node device or drive the at least one node device to change the physical quantity applied on the garment.

For possible embodiments, the smart wearable device can be a smart watch, a smart bracelet, a smart anklet, a smart belt, a smart glasses, a smart voice device or a smart necklace.

In addition, the present invention further provides a smart wearable assembly including:

a smart garment as described above; and

a smart wearable device having a communication interface for coupling with the connection interface to obtain the sensed information of the at least one node device, or drive the at least one node device to change the physical quantity applied on the garment.

For possible embodiments, the smart wearable device can be a smart watch, a smart bracelet, a smart anklet, a smart belt, a smart glasses, a smart voice device or a smart necklace.

For possible embodiments, the smart glasses has at least one function selected by a group consisting of an augmented reality function and a virtual reality function to provide an auxiliary training function or an interaction function or virtual interaction function.

For possible embodiments, the smart wearable device uses an application program to provide an action judgement function to determine whether a user is in a state of standing, sitting, lying or exercising during a predetermined time, or provide a precise action comparison function to assist a user in training for a specific exercise.

### BRIEF DESCRIPTION OF THE DRAWINGS

FIG. 1 is a schematic diagram of a smart wearable assembly according to one embodiment of the present invention.

FIG. 2 is a schematic diagram of a smart wearable assembly according to another embodiment of the present invention.

FIG. 3 is a schematic diagram of a smart wearable assembly according to still another embodiment of the present invention.

### DETAILED DESCRIPTION OF THE PREFERRED EMBODIMENTS

To make it easier for our examiner to understand the objective of the invention, its structure, innovative features, and performance, we use preferred embodiments together with the accompanying drawings for the detailed description of the invention.

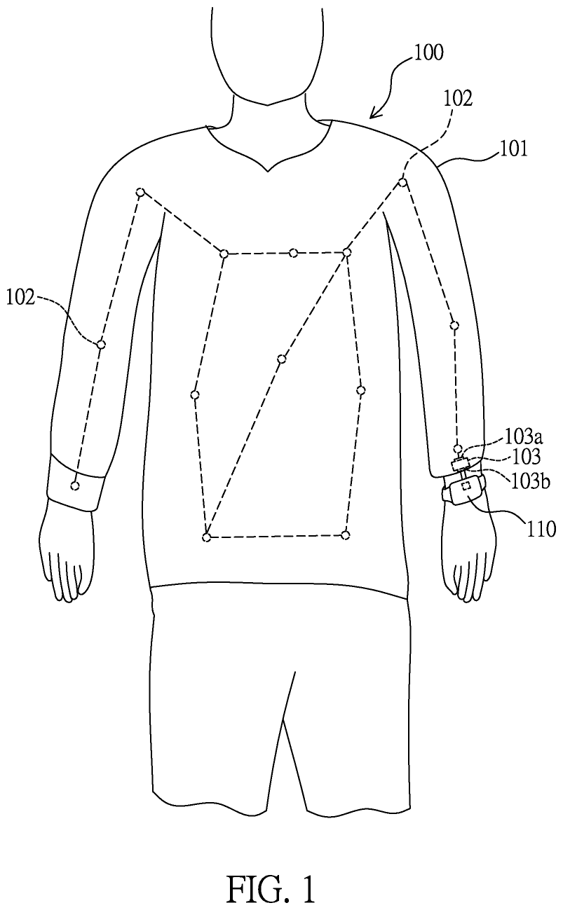

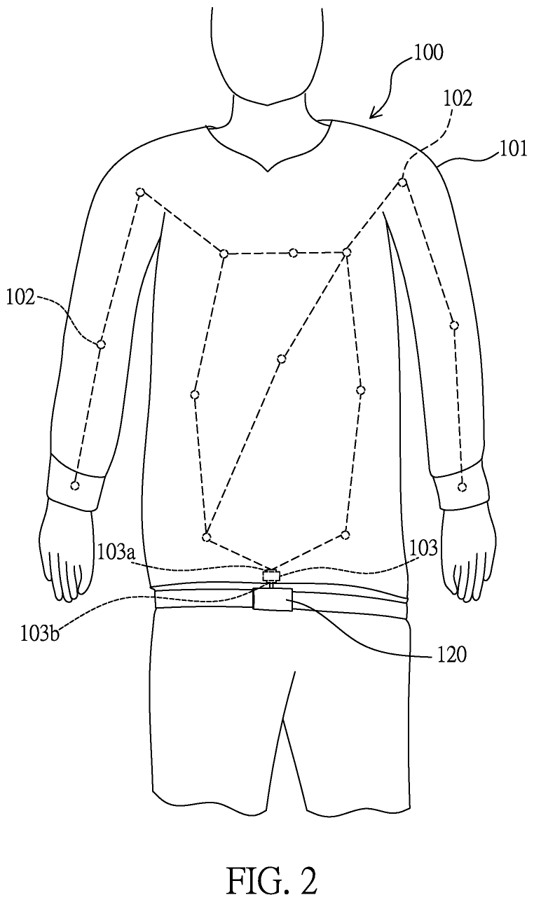

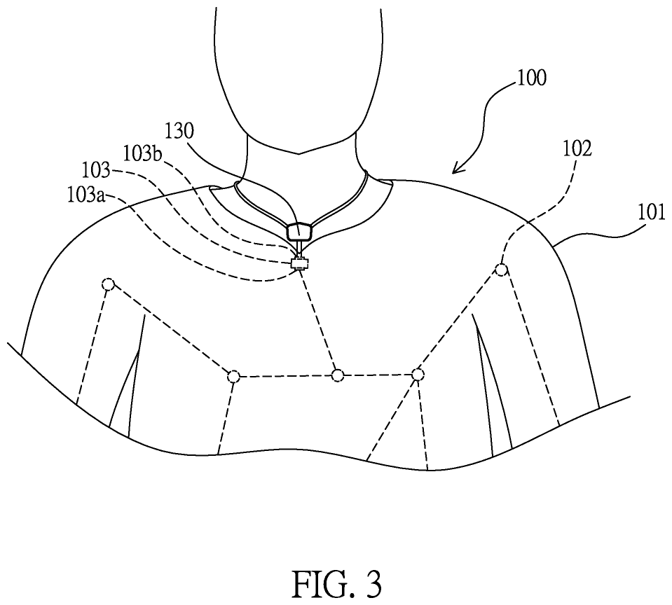

Please refer to FIG. 1-3, which are schematic diagrams of three embodiments of the smart wearable assembly of the present invention, where, the smart wearable assembly shown in FIG. 1 includes a smart garment 100 and a smart watch 110; the smart wearable assembly shown in FIG. 2 includes a smart garment 100 and a smart belt 120; and the smart wearable assembly shown in FIG. 3 includes a smart garment 100 and a smart necklace 130.

As shown in FIG. 1, the smart garment 100 includes a garment 101, a plurality of node devices 102, and a connecting device 103.

The plurality of node devices 102 are distributed on the garment 101, and the connecting device 103 is also disposed on the garment 101. The connecting device 103 has a connecting port 103a electrically coupled with the plurality of node devices 102, and a connection interface 103b for coupling with the smart watch 110 (or smart bracelet or mobile device), so that the smart watch 110 (or smart bracelet or mobile device) can utilize the connection interface 103b to obtain sensed information of the plurality of node devices 102, or drive the plurality of node devices 102 to change a physical quantity applied on the garment 101. In addition, the connection interface 103b can be a wired interface or a wireless interface (for example but not limited to a WiFi interface); the sensed information can include position, heart rate, blood pressure, temperature, or acceleration; and the physical quantity can include heat or pressure.

As shown in FIG. 2, the smart garment 100 includes a garment 101, a plurality of node devices 102, and a connecting device 103. The plurality of node devices 102 are distributed on the garment 101, and the connecting device 103 is also disposed on the garment. The connecting device 103 has a connecting port 103a electrically coupled with the plurality of node devices 102, and a connection interface 103b for coupling with the smart belt 120 (or mobile device), so that the smart belt 120 (or mobile device) can utilize the connection interface 103b to obtain the sensed information of the plurality of node devices 102, or drive the plurality of node devices 102 to change a physical quantity applied on the garment 101. In addition, the connection interface 103b can be a wired interface or a wireless interface (for example but not limited to a WiFi interface); the sensed information can include position, heart rate, blood pressure, temperature, or acceleration; and the physical quantity can include heat or pressure.

As shown in FIG. 3, the smart garment 100 includes a garment 101, a plurality of node devices 102, and a connecting device 103. The plurality of node devices 102 are distributed on the garment 101, and the connecting device 103 is also disposed on the garment. The connecting device 103 has a connecting port 103a electrically coupled with the plurality of node devices 102, and a connection interface 103b for coupling with the smart necklace 130 (or mobile device), so that the smart necklace 130 (or mobile device) can utilize the connection interface 103b to obtain the sensed information of the plurality of node devices 102, or drive the plurality of node devices 102 to change a physical quantity applied on the garment 101. In addition, the connection interface 103b can be a wired interface or a wireless interface (for example but not limited to a WiFi interface); the sensed information can include position, heart rate, blood pressure, temperature, or acceleration; and the physical quantity can include heat or pressure.

Besides, for possible embodiments, the garment 101 can include at least one element of the group consisting of a top, pants, a jumpsuit, a glove, a sock, a hat, a headgear, and a coat. Additionally, for possible embodiments, the node device 102 can be a fixed device or a detachable device.

In addition, for possible embodiments, the smart wearable device of the present invention may be a smart watch, a smart bracelet, a smart anklet, a smart belt, a smart glasses, a smart voice control device or a smart necklace, where the smart glasses may have an augmented reality function, a virtual reality function, or both an augmented reality function and a virtual reality function to provide an auxiliary training function or an interaction function or virtual interaction function. In addition, the smart wearable device of the present invention can use an application program (APP) to provide an action judgment function to determine whether the user is standing, sitting, lying or exercising (for example, cycling, walking, running or swimming) during a predetermined time, or provide a precise action comparison function to assist the user in training for a particular exercise (for example, basketball, yoga, or strength training).

In addition, both the node device 102 and the connection device 103 have a waterproof function.

In addition, the smart watch 110, the smart belt 120, and the smart necklace 130 can communicate with an external information processing device in a wired or wireless manner.

In addition, although the present invention employs a plurality of node devices 102 in the embodiments of FIGS. 1, 2, and 3, the present invention is not limited thereto. In some simple applications, the number of the node devices 102 may be simplified to one. That is, the number of the node devices 102 of the present invention is at least one.

Thus, it can be seen from the description above that the present invention has the following advantages:

1. The smart wearable assembly of the present invention can make a wearable device (smart watch 110, smart belt 120 or smart necklace 130) removable by installing a connecting device 103 on the smart garment 100, so as to avoid damaging the wearable device when the smart garment 100 is washed.

2. The smart wearable assembly of the present invention can greatly enhance the wearing comfort of the smart garment by disposing units of complicated structure or large volume (such as a calculation unit, a battery unit, a storage unit, and the like) in a wearable device, and disposing the sensing nodes having small volume (causing less uncomfortable feeling) in the smart garment.

3. The smart wearable assembly of the present invention is helpful in resources conservation and capable of providing expandability by disposing units of complicated structure or large volume (such as a calculation unit, a battery unit, a storage unit, and the like) in a wearable device, so that the user can use a wearable device to match with multiple smart garments containing less complex units (for example, sensing nodes). In contrast, in the prior art, if a user needs multiple sets of smart garment, as a complete circuit structure including a computing unit, a battery unit, a storage unit and sensing node units is generally installed in each set of smart garment, there will be redundancy of resources to increase the cost substantially.

4. The smart wearable assembly of the present invention can sense position, heart rate, blood pressure, temperature or acceleration by the smart garment 100 for monitoring a wearer's exercise history or body condition.

5. The smart wearable assembly of the present invention can provide a function of relaxing, protecting or warming the body of a wearer by applying heat or pressure to the wearer by the smart garment 100.

While the invention has been described by way of example and in terms of preferred embodiments, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

In summation of the above description, the present invention herein enhances the performance over the conventional structure and further complies with the patent application requirements and is submitted to the Patent and Trademark Office for review and granting of the commensurate patent rights.

What is claimed is:

1. A smart garment comprising:
a garment;
at least one node device distributed on the garment, and the node device being a fixed device or a detachable device; and
a connecting device disposed on the garment and having a connecting port electrically coupled to the at least one node device, and a connection interface for coupling to a wearing device or a mobile device so that the wearing device or the mobile device can utilize the connection interface to obtain sensed information of the at least one node device, or drive the at least one node device to change a physical quantity applied on the garment.

2. The smart garment of claim 1, wherein the sensed information includes at least one element of a group consisting of position, heart rate, blood pressure, temperature, and acceleration.

3. The smart garment of claim 1, wherein the physical quantity includes at least one element of a group consisting of heat and pressure.

4. The smart garment of claim 1, wherein the connection interface is a wired interface or a wireless interface.

5. The smart garment of claim 1, wherein the garment includes at least one element of a group consisting of a top, pants, a jumpsuit, a glove, a sock, a hat, a headgear, and a coat.

6. A smart wearable device having a communication interface coupled to the connection interface of the smart device of claim 1 to obtain the sensed information of the at least one node device or drive the at least one node device to change the physical quantity applied on the garment, wherein the smart wearable device is selected from a group consisting of a smart watch, a smart bracelet, a smart anklet, a smart belt, a smart glasses, a smart voice control device and a smart necklace.

7. A smart wearable device having a communication interface coupled to the connection interface of the smart device of claim 2 to obtain the sensed information of the at least one node device or drive the at least one node device to change the physical quantity applied on the garment, wherein the smart wearable device is selected from a group consisting of a smart watch, a smart bracelet, a smart anklet, a smart belt, a smart glasses, a smart voice control device and a smart necklace.

8. A smart wearable device having a communication interface coupled to the connection interface of the smart device of claim 3 to obtain the sensed information of the at least one node device or drive the at least one node device to change the physical quantity applied on the garment, wherein the smart wearable device is selected from a group consisting of a smart watch, a smart bracelet, a smart anklet, a smart belt, a smart glasses, a smart voice control device and a smart necklace.

9. A smart wearable device having a communication interface coupled to the connection interface of the smart device of claim 4 to obtain the sensed information of the at least one node device or drive the at least one node device to change the physical quantity applied on the garment, wherein the smart wearable device is selected from a group consisting of a smart watch, a smart bracelet, a smart anklet, a smart belt, a smart glasses, a smart voice control device and a smart necklace.

10. A smart wearable device having a communication interface coupled to the connection interface of the smart device of claim 5 to obtain the sensed information of the at least one node device or drive the at least one node device to change the physical quantity applied on the garment, wherein the smart wearable device is selected from a group consisting of a smart watch, a smart bracelet, a smart anklet, a smart belt, a smart glasses, a smart voice control device and a smart necklace.

11. A smart wearable assembly comprising:
the smart garment as claimed in claim 1; and
a smart wearable device having a communication interface for coupling with the connection interface to obtain the sensed information of the at least one node device, or drive the at least one node device to change the physical quantity applied on the garment, wherein the smart wearable device is selected from a group consisting of a smart watch, a smart bracelet, a smart anklet, a smart belt, a smart glasses, a smart voice device and a smart necklace.

12. A smart wearable assembly comprising:
the smart garment as claimed in claim 2; and
a smart wearable device having a communication interface for coupling with the connection interface to obtain the sensed information of the at least one node device, or drive the at least one node device to change the physical quantity applied on the garment, wherein the smart wearable device is selected from a group consisting of a smart watch, a smart bracelet, a smart anklet, a smart belt, a smart glasses, a smart voice device and a smart necklace.

13. A smart wearable assembly comprising:
the smart garment as claimed in claim 3; and
a smart wearable device having a communication interface for coupling with the connection interface to obtain the sensed information of the at least one node device, or drive the at least one node device to change the physical quantity applied on the garment, wherein the smart wearable device is selected from a group consisting of a smart watch, a smart bracelet, a smart anklet, a smart belt, a smart glasses, a smart voice device and a smart necklace.

14. A smart wearable assembly comprising:
the smart garment as claimed in claim 4; and
a smart wearable device having a communication interface for coupling with the connection interface to obtain the sensed information of the at least one node device, or drive the at least one node device to change the physical quantity applied on the garment, wherein the smart wearable device is selected from a group consisting of a smart watch, a smart bracelet, a smart anklet, a smart belt, a smart glasses, a smart voice device and a smart necklace.

15. A smart wearable assembly comprising:
the smart garment as claimed in claim 5; and
a smart wearable device having a communication interface for coupling with the connection interface to obtain the sensed information of the at least one node device, or drive the at least one node device to change the physical quantity applied on the garment, wherein the smart wearable device is selected from a group consisting of a smart watch, a smart bracelet, a smart anklet, a smart belt, a smart glasses, a smart voice device and a smart necklace.

16. The smart wearable assembly of claim 11, wherein the smart glasses has at least one function selected by a group consisting of an augmented reality function and a virtual reality function to provide an auxiliary training function or an interaction function or virtual interaction function; or the smart wearable device uses an application program to provide an action judgement function to determine whether a user is in a state of standing, sitting, lying or exercising during a predetermined time, or provide a precise action comparison function to assist a user in training for a specific exercise; or the smart glasses has at least one function selected by a group consisting of an augmented reality function and a virtual reality function to provide an auxiliary training function or an interaction function or virtual interaction function, and the smart wearable device uses an application program to provide an action judgement function to determine whether a user is in a state of standing, sitting, lying or exercising during a predetermined time, or provide a precise action comparison function to assist a user in training for a specific exercise.

17. The smart wearable assembly of claim 12, wherein the smart glasses has at least one function selected by a group consisting of an augmented reality function and a virtual reality function to provide an auxiliary training function or an interaction function or virtual interaction function; or the smart wearable device uses an application program to provide an action judgement function to determine whether a user is in a state of standing, sitting, lying or exercising during a predetermined time, or provide a precise action comparison function to assist a user in training for a specific exercise; or the smart glasses has at least one function selected by a group consisting of an augmented reality function and a virtual reality function to provide an auxiliary training function or an interaction function or virtual interaction function, and the smart wearable device uses an application program to provide an action judgement function to determine whether a user is in a state of standing, sitting, lying or exercising during a predetermined time, or provide a precise action comparison function to assist a user in training for a specific exercise.

18. The smart wearable assembly of claim 13, wherein the smart glasses has at least one function selected by a group consisting of an augmented reality function and a virtual reality function to provide an auxiliary training function or an interaction function or virtual interaction function; or the smart wearable device uses an application program to provide an action judgement function to determine whether a user is in a state of standing, sitting, lying or exercising during a predetermined time, or provide a precise action comparison function to assist a user in training for a specific exercise; or the smart glasses has at least one function selected by a group consisting of an augmented reality function and a virtual reality function to provide an auxiliary training function or an interaction function or virtual interaction function, and the smart wearable device uses an application program to provide an action judgement function to determine whether a user is in a state of standing, sitting, lying or exercising during a predetermined time, or provide a precise action comparison function to assist a user in training for a specific exercise.

19. The smart wearable assembly of claim 14, wherein the smart glasses has at least one function selected by a group consisting of an augmented reality function and a virtual reality function to provide an auxiliary training function or an interaction function or virtual interaction function; or the smart wearable device uses an application program to provide an action judgement function to determine whether a user is in a state of standing, sitting, lying or exercising during a predetermined time, or provide a precise action comparison function to assist a user in training for a specific exercise; or the smart glasses has at least one function selected by a group consisting of an augmented reality function and a virtual reality function to provide an auxiliary training function or an interaction function or virtual interaction function, and the smart wearable device uses an application program to provide an action judgement function to determine whether a user is in a state of standing, sitting, lying or exercising during a predetermined time, or provide a precise action comparison function to assist a user in training for a specific exercise.

20. The smart wearable assembly of claim 15, wherein the smart glasses has at least one function selected by a group consisting of an augmented reality function and a virtual reality function to provide an auxiliary training function or an interaction function or virtual interaction function; or the smart wearable device uses an application program to provide an action judgement function to determine whether a user is in a state of standing, sitting, lying or exercising during a predetermined time, or provide a precise action comparison function to assist a user in training for a specific exercise; or the smart glasses has at least one function selected by a group consisting of an augmented reality function and a virtual reality function to provide an auxiliary training function or an interaction function or virtual interaction function, and the smart wearable device uses an application program to provide an action judgement function to determine whether a user is in a state of standing, sitting, lying or exercising during a predetermined time, or provide a precise action comparison function to assist a user in training for a specific exercise.
